(19) United States
(12) Patent Application Publication
(10) Pub. No.: US 2005/0171203 A1
(43) Pub. Date: Aug. 4, 2005

(54) Pregabalin composition

(75) Inventors: Hans Richard Meyer-Wonnay, Emmendingen (DE); Michael Schneider, Denzlingen (DE)

Correspondence Address: WARNER-LAMBERT COMPANY, 2800 PLYMOUTH RD, ANN ARBOR, MI, 48105, US

(73) Assignee: Pfizer Inc.

(21) Appl. No.: 11/016,511

(22) Filed: Dec. 17, 2004

(30) Foreign Application Priority Data
Dec. 18, 2003 (EP) 03 029 117.3

(51) Int. Cl. A61K 31/195

(52) U.S. Cl. 514/561

(57) ABSTRACT

A chemically and physically stable, orally administrable aqueous pharmaceutical composition containing pregabalin is described. The liquid composition includes at least one preservative, at least one taste-masking agent, and an optional viscosity control agent. The liquid pharmaceutical composition has a pH of at least about 5.5 but not greater than about 7.0.

### CROSS-REFERENCE TO RELATED APPLICATION

This application claims the benefit of European Patent Application No. 03 029 117.3, filed Dec. 18, 2003.

### BACKGROUND OF THE INVENTION

1. Field of the Invention

This invention relates to liquid pharmaceutical compositions containing pregabalin, which are suitable for oral administration.

2. Discussion

Orally administrable liquid pharmaceutical compositions are attractive dosage forms for treating children and elderly patients. Liquid pharmaceutical compositions are easy to swallow, and if formulated appropriately, have an appealing taste, which may improve patient compliance with a prescribed dosing regimen. In addition, when compared to solid dosage forms, liquid pharmaceutical formulations provide better individualized dosing, which may be important when treating disparate patient populations, such as children and the elderly.

Pregabalin, or (S)-(+)-3-aminomethyl-5-methyl-hexanoic acid, binds to the calcium channel alpha-2-delta (α2δ) subunit and is related to the endogenous inhibitory neurotransmitter γ-aminobutyric acid (GABA), which is involved in the regulation of brain neuronal activity. Pregabalin exhibits anti-seizure activity, as discussed in U.S. Pat. No. 5,563,175 to R. B. Silverman et al., and is thought to be useful for treating, among other conditions, pain, physiological conditions associated with psychomotor stimulants, inflammation, gastrointestinal damage, alcoholism, insomnia, and various psychiatric disorders, including mania and bipolar disorder.

Peroral liquid pharmaceutical compositions containing pregabalin present numerous challenges to formulators. Pregabalin has a strong bitter taste. As a result, any pediatric preparation would likely require taste masking. But pregabalin's high aqueous solubility (32.1 mg/mL) makes taste masking difficult. Furthermore, like gabapentin, pregabalin is a γ-amino acid, which under normal storage conditions and in the presence of water may undergo intramolecular cyclization to form the lactam, 4-isobutyl-pyrrolidin-2-one. See, e.g., WO 99/10186 and WO 99/59573, both to A. Aomatsu. Although it is known that the non-active components of the composition may affect lactam formation, it is difficult to predict which excipients or adjuvants may lead to undesirable lactam formation.

### SUMMARY OF THE INVENTION

The present invention provides a stable liquid pharmaceutical composition for oral administration. The composition comprises pregabalin or a pharmaceutically acceptable complex or salt of pregabalin. The liquid pharmaceutical composition also includes water, at least one preservative, at least one taste-masking agent, and an optional viscosity control agent. The liquid pharmaceutical composition has a pH of at least about 5.5 but not greater than about 7.0.

### DETAILED DESCRIPTION

Unless otherwise indicated, this disclosure uses the following definitions.

“Pharmaceutically acceptable” refers to substances, which are within the scope of sound medical judgment, suitable for use in contact with the tissues of patients without undue toxicity, irritation, allergic response, and the like, commensurate with a reasonable benefit-to-risk ratio, and effective for their intended use.

“Treating” refers to reversing, alleviating, inhibiting the progress of, or preventing a disorder or condition to which such term applies, or to preventing one or more symptoms of such disorder or condition.

“Treatment” refers to the act of “treating” as defined immediately above.

“About,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

“Solvate” describes a molecular complex comprising pregabalin and a stoichiometric amount of one or more pharmaceutically acceptable solvent molecules (e.g., ethanol).

“Hydrate” describes a solvate comprising pregabalin and a stoichiometric amount of water.

As noted above, the peroral liquid pharmaceutical composition comprises pregabalin, which is dissolved or dispersed in water, and includes at least one preservative, at least one taste-masking agent, and one or more optional viscosity control agents. Pregabalin is generally present in the liquid pharmaceutical composition at a concentration of at least about 10 mg/mL and is more typically present at a concentration of at least about 15 mg/mL.

Pregabalin may be prepared using known methods. In some of these methods, a racemic mixture of 3-aminomethyl-5-methyl-hexanoic acid is synthesized and subsequently resolved into its R- and S-enantiomers. Such methods are described in U.S. Pat. No. 5,563,175 to R. B. Silverman et al., U.S. Pat. No. 6,046,353 to T. M. Grote et al., U.S. Pat. No. 5,840,956 to T. M. Grote et al., U.S. Pat. No. 5,637,767 to T. M. Grote et al., U.S. Pat. No. 5,629,447 to B. K. Huckabee & D. M. Sobieray, and U.S. Pat. No. 5,616,793 to B. K. Huckabee & D. M. Sobieray. In each of these methods, the racemate is reacted with a chiral acid (a resolving agent) to form a pair of diastereoisomeric salts, which are separated by known techniques, such as fractional crystallization and chromatography. In other methods, pregabalin is synthesized directly using a chiral auxiliary, (4R,5S)-4-methyl-5-phenyl-2-oxazolidinone. See, e.g., U.S. Pat. Nos. 6,359,169, 6,028,214, 5,847,151, 5,710,304, 5,684,189, 5,608,090, and 5,599,973, all to Silverman et al. In still other methods, pregabalin is prepared via asymmetric hydrogenation of a cyano-substituted olefin to produce a chiral cyano precursor of (S)-3-aminomethyl-5-methyl hexanoic acid, which is subsequently reduced to yield pregabalin. See U.S. Patent Application 2003/0212290 A1 to Burk et al.

When preparing the peroral liquid pharmaceutical composition, one may use any pharmaceutically acceptable form of pregabalin, including without limitation, its free form (zwitterion), and its pharmaceutically acceptable complexes, salts, solvates, hydrates, and polymorphs. Salts include, without limitation, acid addition salts and base addition salts, including hemisalts.

Pharmaceutically acceptable acid addition salts may include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Potentially useful salts include acetate, aspartate, benzoate, chlorobenzoate, methylbenzoate, dinitrobenzoate, besylate, bicarbonate, carbonate, bisulfate, sulfate, pyrosulfate, bisulfite, sulfite, borate, camsylate, caprylate, citrate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, isobutyrate, lactate, malate, maleate, malonate, mandelate, mesylate, methylsulfate, naphthylate, 2-napsylate, nicotinate, nitrate, orotate, oxalate, palmitate, pamoate, phosphate, hydrogen phosphate, dihydrogen phosphate, metaphosphate, pyrophosphate, phthalate, propionate, saccharate, sebacate, stearate, suberate, succinate, tartrate, tosylate, trifluoroacetate, and the like.

Pharmaceutically acceptable base salts may include nontoxic salts derived from bases, including metal cations, such as an alkali or alkaline earth metal cation, as well as amines. Examples of potentially useful salts include, without limitation, aluminum, arginine, N,N′-dibenzylethylenediamine, calcium, chloroprocaine, choline, diethanolamine, diethylamine, dicyclohexylamine, ethylenediamine, glycine, lysine, magnesium, N-methylglucamine, olamine, potassium, procaine, sodium, tromethamine, zinc, and the like. For a discussion of useful acid addition and base salts, see S. M. Berge et al., J. of Pharm. Sci., 66: 1-19 (1977); see also Stahl and Wermuth, Handbook of Pharmaceutical Salts: Properties, Selection, and Use (2002).

The pharmaceutically acceptable salts of pregabalin may be prepared by reacting its free (or zwitterionic) form with a desired acid or base; by removing an acid- or base-labile protecting group from a suitable precursor of pregabalin; by ring-opening a suitable cyclic (lactam) precursor using a desired acid or base; or by converting one salt of pregabalin to another by reaction with an appropriate acid or base or by contact with a suitable ion exchange column. All of these transformations are typically carried out in a solvent. The resulting salt may precipitate out and be collected by filtration or may be recovered by evaporation of the solvent. The degree of ionization in the resulting salt may vary from completely ionized to almost non-ionized.

Pregabalin may exist in both unsolvated and solvated forms and as other types of complexes besides salts. Useful complexes include clathrates or drug-host inclusion complexes where the drug and host are present in stoichiometric or non-stoichiometric amounts. Useful complexes of pregabalin may also contain two or more organic, inorganic, or organic and inorganic components in stoichiometric or non-stoichiometric amounts. The resulting complexes may be ionized, partially ionized, or non-ionized. For a review of such complexes, see J. K. Haleblian, J. Pharm. Sci. 64(8): 1269-88 (1975).

Useful forms of pregabalin include all of its polymorphs and crystal habits, as well as its R-enantiomer and racemic mixtures of pregabalin and its R-enantiomer.

Useful forms of pregabalin would also include pharmaceutically acceptable isotopically labeled compounds in which one or more atoms are replaced by atoms having the same atomic number, but an atomic mass or mass number different from the atomic mass or mass number that predominates in nature. Examples of isotopes suitable for inclusion in pregabalin include isotopes of hydrogen (²H and ³H), carbon (¹¹C, ¹³C and ¹⁴C), and nitrogen (¹³N and ¹⁵N). Isotopically labeled forms of pregabalin may generally be prepared by conventional techniques known to those skilled in the art.

As noted above, the liquid pharmaceutical composition has a pH in the range of about 5.5 to about 7.0, inclusive, preferably in the range of about 5.5 to about 6.5, inclusive, and more preferably in the range of about 5.8 to about 6.2, inclusive. These pH ranges appear to reduce or prevent lactam formation during the preparation of the liquid composition, as well as during storage of the composition for up two years under ICH conditions (25° C. at 60% RH, 30° C. at 60% RH, and 40° C. at 75% RH) and under refrigerated conditions (2° C. to 8° C., inclusive). At a pH below about 5.5 or above about 7.0, lactam formation in the composition appears to be unacceptable (i.e., greater than about 0.5% based on weight); maintaining a pH between about 5.8 and about 6.2, inclusive, appears to minimize lactam formation. During preparation of the liquid composition, pharmaceutically acceptable acids or bases may be added to adjust the pH to the desired value and conventional buffers (e.g. citrate buffers) may be added to maintain the pH within the desired ranges recited above.

The peroral liquid pharmaceutical composition includes one or more preservatives to reduce or prevent microbial growth in multiple dose containers. The choice of preservative depends on a number of criteria. Desired properties, include, but are not limited to, adequate antimicrobial activity between pH 5.5 and 7.0; minimal affect on composition pH; minimal detrimental affect on flavor; suitable for peroral delivery; sufficient solubility in storage at 2° C. to 8° C.; unreactive with pregabalin; unreactive with other formulation components; and stable for two years under ICH conditions. With the exception of antimicrobial properties, these criteria also apply to the other excipients in the liquid pharmaceutical preparation.

As can be seen in the Examples, a number of common preservatives do not meet these criteria. For example, the use of sorbic acid with pregabalin resulted in discoloration of the solution and unacceptable shift in pH to 5.0, which would appear to favor lactam formation. As noted in the Examples, glycerol does not comply with the requirements of the current edition of European Pharmacopoeia (5th ed., 2004). Other common preservatives, such as ethanol, are unsuitable for use in pediatric formulations.

Although a number of the more common preservatives were unacceptable, various parabens (e.g., alkyl-para-hydroxybenzoates) were found to be useful antimicrobial agents in peroral liquid pharmaceutical compositions containing pregabalin. Useful parabens include, without limitation, methylparaben, ethylparaben, propylparaben, butylparaben, and the like, including pharmaceutically acceptable salts thereof, either alone or in combination. Thus, the peroral liquid pharmaceutical composition may include methylparaben and ethylparaben in which, for example, methylparaben is present at a concentration of at least about 2 mg/mL and ethylparaben is present at a concentration of at least about 0.5 mg/mL. Useful paraben combinations include those in which the amount of methylparaben is at least about 3 times the amount of ethylparaben, but not more than about 5 times the amount of ethyl paraben based on weight.

The peroral liquid pharmaceutical composition also includes at least one taste-masking agent. Taste-masking agents include sweetening agents and flavoring agents, which may be used alone or in combination. Although pediatric formulations generally avoid the use of cariogenic sugars as sweetening agents, the common non-cariogenic sugars xylitol and glycerol precipitated out of solution under storage conditions at 5° C., which rendered them unsuitable for use in the liquid pharmaceutical formulation. Subsequent testing indicated that sodium saccharin remained in solution at 5° C. and was compatible with the other components in the formulation. Sodium saccharin may be present in the liquid pharmaceutical composition at a concentration of at least about 0.5 mg/mL, though more typically, it is present at a concentration of at least about 2 mg/mL.

In addition to the sweetening agent, the liquid pharmaceutical composition may include a flavoring agent to mask the bitter taste of pregabalin. Useful flavoring agents include various fruit flavors (e.g., orange, cherry, strawberry, grape, etc.) and mint flavors, which like the preservative, do not react with pregabalin during preparation of the liquid pharmaceutical composition or during storage. Unreactive flavoring agents include those that lack a carbonyl group (i.e., aldehyde and keto groups). Useful flavoring agents thus include Strawberry Flavor 207420, which is available from HAARMANN & REIMER, and Orange Flavor 9/055600, which is available from DRAGOCO.

The liquid pharmaceutical composition may optionally include a viscosity control agent to raise the composition's viscosity. Increasing the viscosity improves the handling of the composition and appears to improve the taste of the pharmaceutical preparation. Useful viscosity control agents include hydroxyethylcellulose, xanthan gum, and the like, which may be used separately or in combination. The viscosity control agent may be used at a concentration of at least about 2 mg/mL up to a concentration of about 5 mg/mL.

The peroral liquid pharmaceutical composition containing pregabalin may be prepared as described below in Examples 6 to 9. The resulting syrup-like pharmaceutical compositions may be filled into single-dose or multiple-dose containers. A double-sachet of coated aluminium foil containing two half-doses may be useful as a single dose container. Multiple dose containers permit variable volume dosing and may be provided with an appropriate dosage aid (e.g., graduated cup or pipette). Multiple dose containers may be glass or plastic bottles fitted with a childproof closure. Useful closures include those lined with polyethylene foam.

### EXAMPLES

The following examples are intended to be illustrative and non-limiting, and represent specific embodiments of the present invention.

### Example 1 to Example 5

TABLE 1 lists representative results of antimicrobial efficacy testing for liquid pharmaceutical compositions containing pregabalin and various preservatives, including sorbic acid, methylparaban, ethylparaban, and glycerol. In all of the examples shown in TABLE 1, the pregabalin concentration is 15 mg/mL and sufficient amount of purified water is added to give the batch size noted. In TABLE 1, the annotations “Meets Ph. Eur.” and “Fails Ph. Eur.” refer to whether the test results meet or fail the microbial limit test as defined in the current edition of European Pharmacopoeia (5th ed., 2004): 1000 bacteria per g (max); 100 yeasts and molds per g (max); and free of E. coli.

### Example 6 to Example 9

TABLE 2 lists representative liquid pharmaceutical compositions containing pregabalin, which undergo stability testing. In all of the examples shown in TABLE 2 a sufficient amount of purified water is added to give a 5 L batch size. Example 6 and Example 7 differ in the flavoring agent used, but are otherwise the same. Example 8 and Example 9 are similar to Example 6, except that they are adjusted to pH 5.5 and to pH 7.0 through the addition of 0.1 N HCl and 0.1 N NaOH, respectively.

For each of Examples 6 to 9, purified and sterile water (about 4.5 L) is placed in a temperature-controlled vessel outfitted with a stirrer. The contents of the vessel are heated to 80° C. Methylparaben (10 g) and ethylparaben (2.5 g) are stirred into the hot water. After a clear solution is obtained, sodium saccharin (5 g) is added. Next, hydroxyethylcellulose (45 g) is added in small portions with vigorous stirring to avoid lumps. The resulting liquid mixture is cooled to 30° C. and pregabalin (75 g) is added in small portions with stirring. After pregabalin is completely dissolved, cooling is continued and the flavoring agent (10 g of Strawberry Flavor or 5 g of Orange Flavor) is added at 25° C. to 30° C. The mixture is stirred for at least 10 minutes to homogeneously disperse each of the flavoring agents. The solution is filtered using a MILLIPORE® membrane filter (cellulose acetate with a pore size of 10 μm). Purified water is added, along with 0.1 N HCl or 0.1 N NaOH (Examples 8 and 9) so that the total batch size is 5 L. The resulting liquid pharmaceutical composition is stored in sealed containers.

Stability tests were performed for each of the peroral liquid pregabalin formulations in Examples 6 to 9. The stability tests were performed upon initial preparation of the compositions and were scheduled to be performed after 1, 3, 6, 12, 18, and 24 months of storage at 5° C., at 25° C. and 60% RH, and at 30° C. and 60% RH. The stability protocol included an examination of the appearance and color of the liquid compositions, along with assays of pregabalin and degradant concentration, solution pH, and antimicrobial activity (at 25° C. only).

After 6 months of storage, the formulations were clear or slightly opaque and were colorless or slightly yellow. In addition, after 6 months storage, all of the batches had no more than 0.1% lactam formation based on weight, and had pH's between 5.4 and 7.0, regardless of whether their initial pH was adjusted or not. Finally, all of the batches met the microbial limits set forth in the current edition of the European Pharmacopoeia.

It should be noted that, as used in this specification and the appended claims, singular articles such as “a,” “an,” and “the,” may refer to one object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patents, patent applications and publications, are incorporated herein by reference in their entirety and for all purposes.

TABLE 1
Antimicrobial Activity of Aqueous Liquid Pharmaceutical Compositions of Pregabalin

| | | | | Storage² | | |
|---|---|---|---|---|---|---|
| Example | Volume L | Component¹ | mg/mL | Months | Results | Remarks |
| 1 | 4 | Sorbic acid | 2 | | Not determined | pH-shift, undissolved particles, |
| | | Xylitol C NF | 200 | | | pink color |
| 2 | 5 | Ethylparaben | 0.3 | 0 | Meets Ph. Eur. | |
| | | Methylparaben | 1.2 | 3 | Fails Ph. Eur | |
| | | Sodium Saccharin | 0.5 | | (A. niger) | |
| 3 | 5 | Ethylparaben | 0.4 | 0 | Meets Ph. Eur. | |
| | | Methylparaben | 1.6 | 1 | Meets Ph. Eur. | |
| | | Saccharin Sodium | 0.5 | 3 | Fails Ph. Eur. | |
| | | Hydroxyethylcellulose 250 HHX | 3 | | (A. niger) | |
| 4 | 5 | Glycerin min. 99% | 400 | 0 | Fails Ph. Eur. | mold |
| | | Saccharin Sodium | 0.6 | 1 | (A. niger, C. albicans) | |
| | | 0.1 N HCl (mL) | 0.01 | | Fails Ph. Eur. | |
| | | | | | (A. niger, C. albicans) | |
| 5 | 5 | Ethylparaben | 0.5 | 0 | Meets Ph. Eur. | |
| | | Methylparaben | 2 | 1 | Meets Ph. Eur. | |
| | | Saccharin Sodium | 1 | 3 | Meets Ph. Eur. | |
| | | Hydroxyethylcellulose 250 HHX | 3 | 6 | Meets Ph. Eur. | |
| | | Strawberry flavor | 2 | | | |

¹Purified water added to give indicated batch volume; pregabalin concentration is 15 mg/mL.
²Stored at 25° C. and 60% relative humidity (RH).

TABLE 2
Aqueous Pregabalin Compositions¹

| | Example | | | |
|---|---|---|---|---|
| Component | 6 | 7 | 8² | 9³ |
| Pregabalin | 15 | 15 | 15 | 15 |
| Methylparaben | 2 | 2 | 2 | 2 |
| Ethylparaben | 0.5 | 0.5 | 0.5 | 0.5 |
| Sodium Saccharin | 1 | 1 | 1 | 1 |
| Hydroxyethylcellulose 250 HHX | 3 | 3 | 3 | 3 |
| Strawberry Flavor 207420 H&R | 2 | | 2 | 2 |
| Orange Flavor 9/055600 DRAGOCO | | 1 | | |

¹Purified water added to give a 5 L batch size.
²0.1 N HCl added to adjust pH to 5.5.
³0.1 N NaOH added to adjust pH to 7.0.

1. A liquid pharmaceutical composition for oral administration comprising:
pregabalin or a pharmaceutically acceptable salt or complex thereof;
water;
at least one preservative;
at least one taste-masking agent; and
an optional viscosity control agent;
wherein the liquid pharmaceutical composition has a pH of at least about 5.5 but not greater than about 7.0.

2. The liquid pharmaceutical composition of claim 1, wherein the liquid pharmaceutical composition has a pH of at least about 5.5 but not greater than about 6.5.

3. The liquid pharmaceutical composition of claim 2, wherein the liquid pharmaceutical composition has a pH of at least about 5.8 but not greater than about 6.2.

4. The liquid pharmaceutical composition of claim 1, wherein pregabalin is present in the liquid pharmaceutical composition at a concentration of at least about 10 mg/mL.

5. The liquid pharmaceutical composition of claim 4, wherein pregabalin is present in the liquid pharmaceutical composition at a concentration of at least about 15 mg/mL.

6. The liquid pharmaceutical composition of claim 1, wherein the preservative comprises one or more parabens, including pharmaceutically acceptable salts thereof.

7. The liquid pharmaceutical composition of claim 1, wherein the preservative comprises methylparaben and ethylparaben and pharmaceutically acceptable salts thereof.

8. The liquid pharmaceutical composition of claim 7, wherein methylparaben, or a pharmaceutically acceptable salt thereof, is present in the liquid pharmaceutical composition at a concentration of at least about 2 mg/mL.

9. The liquid pharmaceutical composition of claim 8, wherein ethylparaben, or a pharmaceutically acceptable salt thereof, is present in the liquid pharmaceutical composition at a concentration of at least about 0.5 mg/mL.

10. The liquid pharmaceutical composition of claim 1, wherein the taste-masking agent comprises a sweetening agent.

11. The liquid pharmaceutical composition of claim 10, wherein the sweetening agent is sodium saccharin.

12. The liquid pharmaceutical composition of claim 11, wherein sodium saccharin is present in the liquid pharmaceutical composition at a concentration of at least about 0.5 mg of per mL.

13. The liquid pharmaceutical composition of claim 11, wherein sodium saccharin is present in the liquid pharmaceutical composition at a concentration of at least about 2 mg/mL.

14. The liquid pharmaceutical composition of claim 1, wherein the taste-masking agent comprises a flavoring agent, the flavoring agent characterized by the absence of carbonyl groups.

15. The liquid pharmaceutical composition of claim 1, wherein the viscosity control agent is hydroxyethylcellulose.
